(19) United States
(12) Patent Application Publication
(10) Pub. No.: US 2016/0030697 A1
(43) Pub. Date: Feb. 4, 2016

(54) APPARATUS, SYSTEM, AND METHOD FOR ENDOTRACHEAL TUBE PLACEMENT

(71) Applicant: University of Maryland, Baltimore, Baltimore, MD (US)

(72) Inventor: Paul Bigeleisen, Baltimore, MD (US)

(21) Appl. No.: 14/813,303

(22) Filed: Jul. 30, 2015

Related U.S. Application Data
(60) Provisional application No. 62/031,194, filed on Jul. 31, 2014.

(51) Int. Cl. A61M 16/08; A61B 1/267; A61M 16/04

(52) U.S. Cl. CPC A61M 16/0833; A61M 16/0447; A61M 16/0486; A61B 1/2676

(57) ABSTRACT

Disclosed are an apparatus, system, and method for endotracheal tube placement that allow for ventilation of the patient throughout the process of placing an endotracheal tube. The apparatus includes a T-shaped adaptor having a first lumen for coaxial connection to an artificial airway and a second lumen that intersects the first lumen for connection to a ventilator. A cap is provided over the distal end of the first lumen, the cap having an opening that may be closed with a flexible plug, blocking air from exiting through the first lumen when the second lumen is attached to a ventilator. The opening is sized to receive a fiber optic bronchoscope and to flexibly seal against the exterior of the fiber optic bronchoscope as it passes through the cap. A line of weakening extends from the opening to the perimeter of the cap, such that after the fiber optic bronchoscope has been advanced to the patient's trachea, the endotracheal tube may be advanced toward the cap, the cap may be split along the line of weakening to allow its removal from both the T-shaped adaptor and the fiber optic bronchoscope, and the endotracheal tube may be advanced over the fiber optic bronchoscope through the first lumen and the supraglottic airway without removal of the T-shaped adaptor, and thus while maintaining ventilation that is supplied through the second lumen of such T-shaped adaptor.

### CROSS-REFERENCE TO RELATED APPLICATION

This application is based upon and claims the benefit of copending U.S. Provisional Patent Application Ser. No. 62/031,194 entitled “Disposable Airway T-Piece,” filed with the U.S. Patent and Trademark Office on Jul. 31, 2014 by the inventor herein, the specification of which is incorporated herein by reference in its entirety.

### FIELD OF THE INVENTION

The present invention relates generally to mechanical ventilation and intubation devices and methods, and more particularly to devices and methods for providing an interface between artificial airway devices and a ventilation machine.

### BACKGROUND OF THE INVENTION

During various surgical procedures and in certain emergency medical situations, it is necessary to provide a patient with an artificial airway, often by placement of an endotracheal tube through the patient's mouth and into their trachea using a laryngoscope. However, in some cases and for a variety of reasons, it may be very difficult to place an endotracheal tube into a patient with a direct laryngoscope. When this situation arises, a fiber optic bronchoscope is often used. In this procedure, an endotracheal tube is loaded onto the bronchoscope, and the bronchoscope is passed through the mouth, past the vocal cords and into the trachea. Once the bronchoscope is placed in the trachea, the endotracheal tube is pushed off of the fiber optic bronchoscope and into the trachea. In many patients, this can be accomplished while the patient is sedated and the patient's airway is anesthetized with local anesthetic. In this setting, the patient maintains oxygenation with spontaneous respiration. However, in some patients, such as children and mentally disabled adults, this task must be accomplished while the patient is apneic under general anesthesia, thus requiring artificial respiration. As the procedure for placing the bronchoscope into the trachea may take as long as five to ten minutes, proper management of such artificial respiration during the process is critical.

In order to maintain artificial respiration during such process, and particularly where general anesthesia is required, traditional techniques have involved the use of a supraglottic airway, an example of which is shown in FIG. 1. A supraglottic airway, shown generally at 10, typically includes a small latex mask 12, typically having an inflatable cuff extending around the perimeter of the mask 12, and configured for placement in the lower pharynx of a patient when necessary to manage a difficult airway. A hollow plastic tube 14 extends from mask 12. A T-adaptor 16 may be provided at the distal end of tube 14, having a first lumen 18 aligned with tube 14 and a second lumen 20 intersecting lumen 18. T-shaped adaptor 16 provides a port for both mechanical ventilation (through lumen 20) and a port for a fiber optic bronchoscope (through lumen 18). Adaptor 16 must be removed from tube 14 in order to place an endotracheal tube through supraglottic airway 10 and into the patient's trachea, thus leaving the patient without ventilation while the endotracheal tube is being placed. This period of apnea can result in life threatening hypoxemia to the patient.

In light of the foregoing limitations of previously known devices and methods, there remains a need in the art for systems and methods that allow for ventilation of a patient throughout the process of placing an endotracheal tube, including in those cases in which a supraglottic airway is required.

### SUMMARY OF THE INVENTION

Disclosed herein are an apparatus, system, and method for endotracheal tube placement that allow for ventilation of the patient throughout the process of placing an endotracheal tube. The apparatus includes a T-shaped adaptor having a first lumen for coaxial connection to an artificial airway, such as a supraglottic airway, and a second lumen that intersects the first lumen for connection to a ventilator. A cap is provided over the distal end of the first lumen, the cap having an opening that may be closed with a flexible plug, blocking air from exiting through the first lumen when the second lumen is attached to a ventilator. The flexible plug is configured for manual removal from the opening in the cap, with the opening being sized to receive a fiber optic bronchoscope and to flexibly seal against the exterior of the fiber optic bronchoscope as it passes through the cap. A line of weakening extends from the opening to the perimeter of the cap. Thus, after the fiber optic bronchoscope has been advanced to the patient's trachea, the endotracheal tube (which is pre-positioned on the fiber optic bronchoscope) may be advanced toward the cap, the cap may be split along the line of weakening to allow its removal from both the T-shaped adaptor and the fiber optic bronchoscope, and the endotracheal tube may be advanced over the fiber optic bronchoscope through the first lumen and the supraglottic airway without removal of the T-shaped adaptor, and thus while maintaining ventilation that is supplied through the second lumen of such T-shaped adaptor.

In accordance with certain aspects of an embodiment of the invention, an apparatus is provided that is configured for interfacing mechanical ventilation with an artificial airway device, the apparatus comprising: a T-connector having a distal end, a proximal end, a first lumen extending from the distal end to the proximal end, and a second lumen extending outward from the first lumen; and a cap removably positioned on the distal end of the T-connector, the cap having an annular top face defining an opening extending through the top face, and a line of weakening along the top face extending from the opening to an outer perimeter of the cap.

In accordance with further aspects of an embodiment of the invention, a system is provided for placing an endoscopic tube in a patient, the system comprising: a supraglottic airway comprising a mask and a flexible hose extending from the mask; a T-connector having a distal end, a proximal end, a first lumen extending from the distal end to the proximal end, and a second lumen extending outward from the first lumen, wherein the proximal end is configured for removable attachment to an end of the flexible hose of the supraglottic airway opposite the mask; and a cap removably positioned on the distal end of the T-connector, the cap having an annular top face defining an opening extending through the top face, and a line of weakening along the top face extending from the opening to an outer perimeter of the cap.

In accordance with still further aspects of an embodiment of the invention, a method is provided for placing an endotracheal tube in a patient, comprising: providing a supraglottic airway comprising a mask and a flexible hose extending from the mask; removably attaching a T-connector to an end of the flexible hose of the supraglottic airway, the T-connector having a distal end, a proximal end, a first lumen extending from the distal end to the proximal end, and a second lumen extending outward from the first lumen; providing a cap removably positioned on the distal end of the T-connector, the cap having an annular top face defining an opening extending through the top face, and a line of weakening along the top face extending from the opening to an outer perimeter of the cap; and communicating a ventilator with the second lumen of said T-connector.

### BRIEF DESCRIPTION OF THE DRAWINGS

The numerous advantages of the present invention may be better understood by those skilled in the art by reference to the accompanying figures in which:

FIG. 1 is a perspective view of a prior art supraglottic airway and T-shaped connector.

FIG. 2 is a perspective view of a T-shaped adaptor in accordance with certain aspects of an embodiment of the invention.

FIG. 3 is a top view of a cap for use with the T-shaped adaptor of FIG. 2.

FIG. 4 is a side view of a plug of the cap of FIG. 3.

FIG. 5 is a perspective view of the T-shaped adaptor and cap of FIGS. 3 and 4 positioned on a supraglottic airway.

FIG. 6 is perspective view of the T-shaped adaptor and cap of FIG. 5 with a fiber optic bronchoscope passed through the cap.

FIG. 7 is a perspective view of the T-shaped adaptor and cap of FIG. 5 with an endotracheal tube passed through the adaptor and the supraglottic airway following removal of the cap.

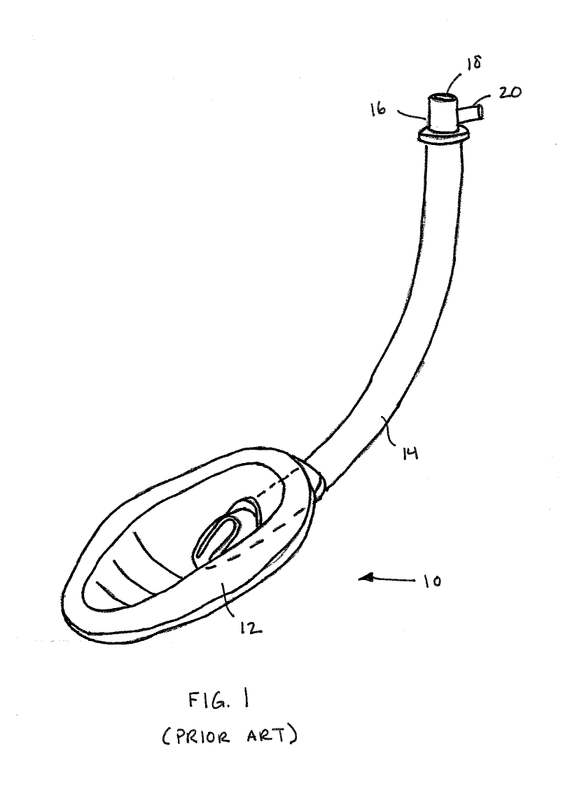

### DETAILED DESCRIPTION OF THE PREFERRED EMBODIMENTS

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

An apparatus, system, and method are described for endotracheal tube placement that allow for ventilation of the patient throughout the process of placing an endotracheal tube. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of a separate part. However, it should be understood that existing laryngeal scopes and ventilation masks could be manufactured to include the features of the invention, including by way of example the adaptor described in detail below. One having ordinary skill in the art would recognize minor changes that would be necessary to adapt the system for different uses. These modifications should be considered part of the invention because they do not deviate from its overall spirit.

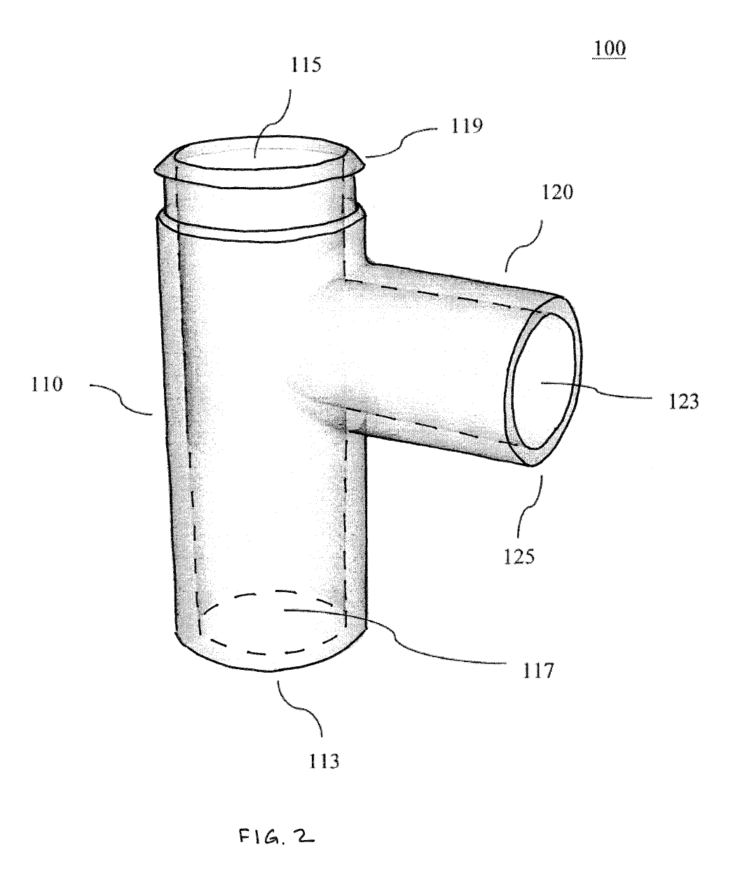

Referring now to the figures, FIG. 2 is a perspective view of the main body of a T-shaped adaptor 100 in accordance with certain aspects of an embodiment of the invention. The body comprises a main elongated shaft 110 with a proximal end 113 and a distal end 115. At least one lumen 117 extends through the length of shaft 110. A second shaft 120 extends outwards from the sidewall of shaft 110. A second lumen 123 communicates with first lumen 117 and extends through the distal end 125 of second shaft 120.

The proximal end 113 of main shaft 110 is configured to be joined to an artificial airway device, such as a laryngeal scope or ventilation mask. In the illustrated embodiment, the proximal end 113 may comprise a bushing that is configured to receive the laryngeal scope or mask. The bushing at proximal end 113 may optionally comprise a separate piece that is configured to swivel to allow the adaptor 100 to rotate while attached to another device. Lumen 117 is preferably sized to receive and allow for the passage of existing endotracheal tubes.

The distal end 115 of main shaft 110 is configured to removably receive a protective cap 200 as discussed in greater detail below with reference to FIG. 3. In certain configurations, distal end 115 of adaptor 100 may include an outwardly flanged lip 119 extending around the perimeter of distal end 115, which lip 119 forms a surface for removably holding a flexible ridge 212 on the underside of cap 200.

Further, the distal end 125 of second shaft 120 is configured to communicate with existing mechanical ventilators (not shown). Second shaft 120 may extend perpendicularly from main shaft 110 of adaptor 100, or may project outwards at any other angle. In certain configurations, distal end 125 of second shaft 120 may comprise a bushing that is configured to receive a hose from a ventilator.

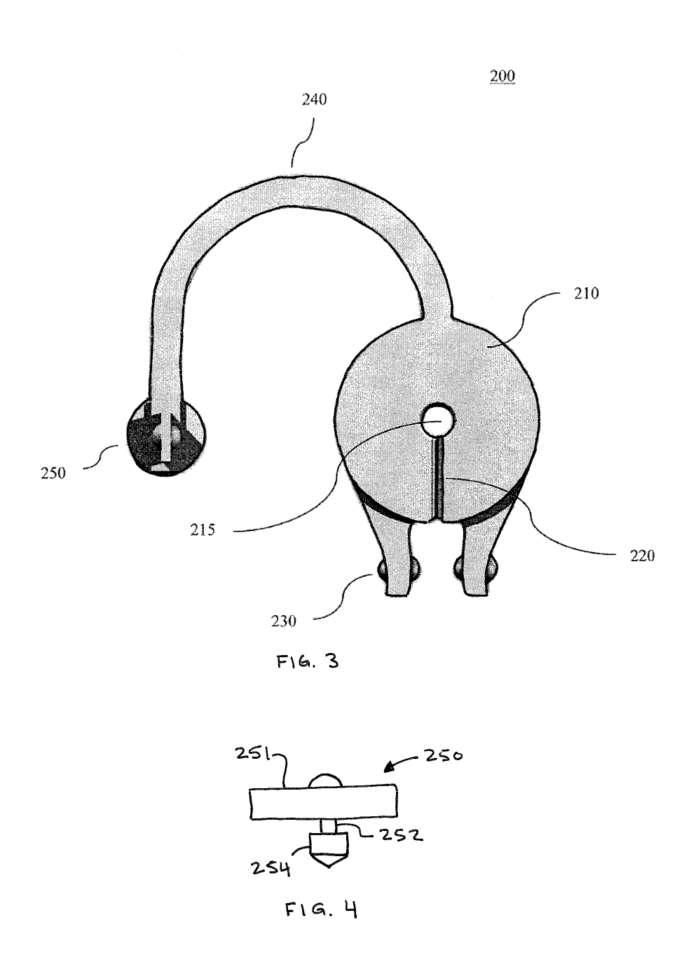

In accordance with further aspects of an embodiment of the invention, FIG. 3 is a top view of a cap 200 for use with T-shaped adaptor 100, and FIG. 4 is a side view of a plug for use with cap 200. Cap 200 has a main body 210 configured to cover and removably attach to the distal end 115 of main shaft 110 of adaptor 100. Main body 210 includes flexible ridge 212 extending around the perimeter of the bottom of main body 210, which flexible ridge may be stretched to extend over lip 119 of adaptor 100 and thereafter return to its original shape so as to hold flexible ridge 212 below the bottom, widest portion of lip 119. Main body 210 is annular, such that it defines an opening 215 that may, by way of non-limiting example, be positioned generally at the center of main body 210. A flexible and compressible plug 250 is provided with protective cap 200, and preferably is attached to main body 210 via a flexible tether 240. Plug 250 is shaped to removably fit within opening 215. As shown in FIG. 4, plug 250 is provided a neck 252 extending downward from the bottom side of a plug body 251, and a head portion 254 at the end of neck 252. Neck 252 is sized to fit within opening 215, with head portion 254 having a diameter larger than opening 215. Thus, head portion 254 may be compressed as it is pushed through opening 215 and, once pushed through, expand to hold plug 250 in place to seal cap 200. In this configuration, if a mechanical respirator is attached to second shaft 120, plug 250 will block air from exiting adaptor 100 through distal end 115, allowing all air to be delivered through proximal end 113 and to any mask or other artificial airway device attached thereto.

Likewise, when ready to insert a fiber optic bronchoscope into the patient's larynx, plug 250 may be pulled upward to remove head portion 254 from opening 215, after which the fiber optic bronchoscope may be inserted into opening 215. Opening 215 is thus sized slightly smaller than a fiber optic bronchoscope to be used with adaptor 100, and as main body 210 is formed of a flexible material, will stretch slightly as the bronchoscope passes through main body 210 and form a seal against the exterior of the bronchoscope as it moves toward and through proximal end 113 of adaptor 100. Opening 215 may be provided in any size that is necessary to ensure a tight seal with a fiber optic bronchoscope that is to be used with adaptor 100, while still allowing the bronchoscope to be pushed through opening 215. In this configuration, if a mechanical respirator is attached to second shaft 120, main body 210 continues to block air from exiting adaptor 100 through distal end 115, thus continuing to allow all air to be delivered through proximal end 113 and to any mask or other artificial airway device attached thereto.

With continued reference to FIG. 3, main body 210 of protective cap 200 is also provided a line of weakening 220, such as a partially perforated portion, a region of thin material, or similarly configured weakened section, extending from opening 215 to the outer perimeter of main body 210. Line of weakening 220 is configured to cause main body 210 to split apart at such line of weakening 220 before any other portion of main body 210. To aid in allowing main body 210 to be split apart along line of weakening 220, one or more tabs 230 are provided extending outward from the exterior perimeter of main body 210. In an exemplary configuration, two such tabs 230 are provided, with one tab positioned on each side of line of weakening 220. Tabs 230 may be pulled away from one another so as to cause main body to split along line of weakening 220, which in turn allows the entirety of protective cap 200 to be removed from adaptor 100 and from the fiber optic bronchoscope extending through lumen 117.

Cap 200, including at least main body 210 and plug 250, is preferably formed of an at least semi-flexible material, preferably latex or polyurethane, in order to ensure that protective cap 200 remains sufficiently flexible to allow its removable fitment around lip 119 and manual insertion and removal of plug 250 from opening 215, while maintaining sufficient rigidity to allow a fiber optic bronchoscope to travel through opening 215 without excessive deformation of cap 200.

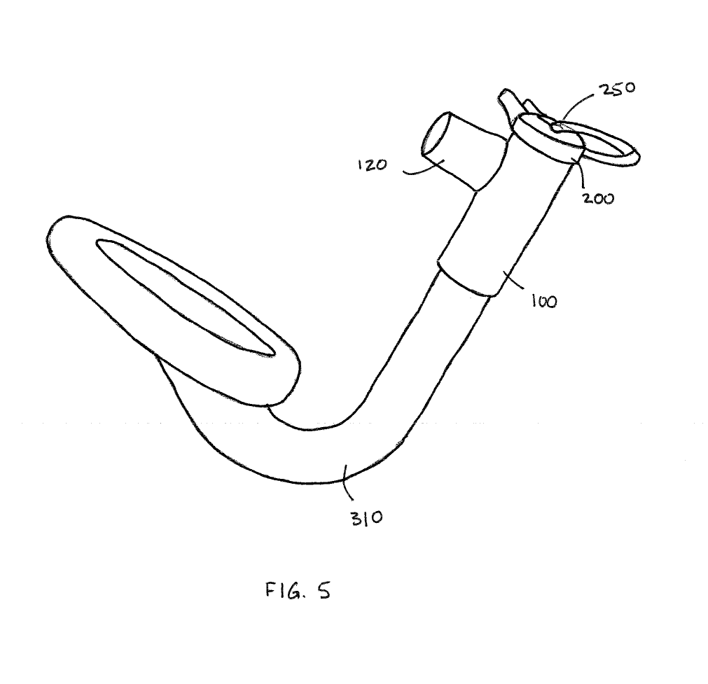

Next, and in accordance with still further aspects of an embodiment of the invention, FIGS. 5 through 7 show a system including adaptor 100 and protective cap 200 attached to an artificial airway device, such as a supraglottic airway 310, and a method of using such system to place an endotracheal tube into a patient's airway. While the system is shown in FIGS. 5 through 7 outside of a patient, in use the supraglottic airway 310 is introduced into the patient after the initiation of general anesthesia and the commencement of mechanical ventilation. Once in place, the proximal end 113 of adaptor 100 is placed on the distal end of supraglottic airway 310. As shown in FIG. 3, protective cap 200 begins on the distal end of the adaptor 100, and plug 250 is used to seal opening 215. Mechanical ventilation (not shown) is introduced through second shaft 120, flowing through second lumen 123 into first lumen 117 and out of adaptor 100 through its proximal end 113 and into supraglottic airway 310.

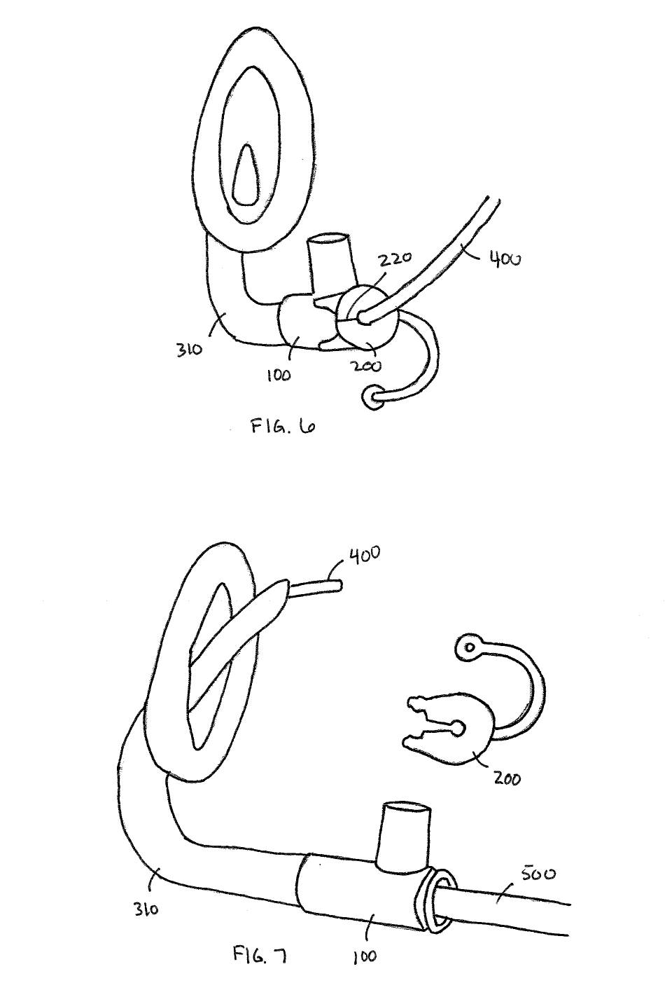

Once the operator is ready to introduce a fiber optic bronchoscope, as shown in FIG. 6, plug 250 is removed from opening 215, and the proximal end of the fiber optic bronchoscope 400 is inserted through opening 215 in cap 200, and is thereafter advanced into tube 14 of supraglottic airway 310 and ultimately out of mask 12. After the fiber optic bronchoscope 400 is in place, and with reference to FIG. 7, tabs 230 on cap 200 are pulled apart causing main body 210 to split apart along line of weakening 220, allowing cap 200 to be entirely removed from adaptor 100 and fiber optic bronchoscope 400 without removal of bronchoscope 400 from the supraglottic airway. An endotracheal tube 500 that has been pre-positioned on fiber optic bronchoscope 400 may then immediately be advanced through the distal end 115 of adaptor 100, down through lumen 117, out of proximal end 113 and into supraglottic airway 310, and ultimately out of mask 12 and into the patient's trachea. As the inner diameter of adaptor 100 is larger than the outer diameter of endotracheal tube 500, air delivered from a ventilator through second lumen 123 of adaptor 100 will continue to flow to the patient during insertion of the endotracheal tube 500. Once the endotracheal tube 500 is in place in the patient, fiber optic bronchoscope 400, and thereafter supraglottic airway 310, may be removed from the patient, leaving the endotracheal tube in place for immediate connection to mechanical ventilation. As mechanical ventilation may be continued throughout this process, the risk of the patient experiencing hypoxemia during placement of the endotracheal tube is significantly reduced.

In the foregoing description, the invention has been described with reference to specific embodiments thereof. It will, however, be evident to those of ordinary skill in the art that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps. Furthermore, the indefinite article “a” or “an” is meant to indicate one or more of the item, element, or step modified by such article. Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. An apparatus configured for interfacing mechanical ventilation with an artificial airway device, comprising:
a T-connector having a distal end, a proximal end, a first lumen extending from said distal end to said proximal end, and a second lumen extending outward from said first lumen; and
a cap removably positioned on said distal end of said T-connector, said cap having an annular top face defining an opening extending through said top face, and a line of weakening along said top face extending from said opening to an outer perimeter of said cap.

2. The apparatus of claim 1, further comprising a flexible plug sized for removable attachment to said cap through said opening.

3. The apparatus of claim 2, further comprising a flexible tether attaching said plug to said cap.

4. The apparatus of claim 1, further comprising at least one pull tab extending from said outer perimeter of said cap and adjacent said line of weakening.

5. The apparatus of claim 1, wherein said opening is configured to receive a proximal end of a fiber optic bronchoscope and to allow a fiber optic bronchoscope to be moved through said opening while preventing air flow between said fiber optic bronchoscope and said opening.

6. The apparatus of claim 5, wherein said first lumen is sized to receive an endotracheal tube.

7. A system for placing an endoscopic tube in a patient, comprising:
a supraglottic airway comprising a mask and a flexible hose extending from said mask;
a T-connector having a distal end, a proximal end, a first lumen extending from said distal end to said proximal end, and a second lumen extending outward from said first lumen, wherein said proximal end is configured for removable attachment to an end of said flexible hose of said supraglottic airway opposite said mask; and
a cap removably positioned on said distal end of said T-connector, said cap having an annular top face defining an opening extending through said top face, and a line of weakening along said top face extending from said opening to an outer perimeter of said cap.

8. The system of claim 7, said second lumen having a distal end and a proximal end, wherein said proximal end of said second lumen intersects said first lumen, and wherein said distal end of said second lumen is configured for removable attachment to a ventilator hose.

9. The system of claim 8, further comprising a ventilator in fluid communication with said distal end of said second lumen.

10. The system of claim 7, further comprising a flexible plug sized for removable attachment to said cap through said opening.

11. The system of claim 10, further comprising a flexible tether attaching said plug to said cap.

12. The system of claim 7, further comprising at least one pull tab extending from said outer perimeter of said cap and adjacent said line of weakening.

13. The system of claim 7, wherein said opening is configured to receive a proximal end of a fiber optic bronchoscope and to allow a fiber optic bronchoscope to be moved through said opening while preventing air flow between said fiber optic bronchoscope and said opening.

14. The system of claim 13, wherein said first lumen is sized to receive an endotracheal tube.

15. A method for placing an endotracheal tube in a patient, comprising:
providing a supraglottic airway comprising a mask and a flexible hose extending from said mask;
removably attaching a T-connector to an end of said flexible hose of said supraglottic airway, said T-connector having a distal end, a proximal end, a first lumen extending from said distal end to said proximal end, and a second lumen extending outward from said first lumen;
providing a cap removably positioned on said distal end of said T-connector, said cap having an annular top face defining an opening extending through said top face, and a line of weakening along said top face extending from said opening to an outer perimeter of said cap; and
communicating a ventilator with said second lumen of said T-connector.

16. The method of claim 15, further comprising the step of passing a proximal end of a fiber optic bronchoscope through said opening in said cap and advancing said proximal end of said fiber optic bronchoscope through said proximal end of said T-connector as air is supplied from said ventilator to said T-connector.

17. The method of claim 16, wherein said opening in said cap is configured to prevent air flow between said fiber optic bronchoscope and said opening as said fiber optic bronchoscope is advanced.

18. The method of claim 16, further comprising the step of tearing said cap along said line of weakening after advancing said fiber optic bronchoscope.

19. The method of claim 18, further comprising the step of removing said cap from said T-connector and said fiber optic bronchoscope while said proximal end of said fiber optic bronchoscope is advanced at least to said mask.

20. The method of claim 19, further comprising the steps of advancing an endotracheal tube over said fiber optic bronchoscope and into a patient's trachea as air is supplied from said ventilator to said T-connector, and removing said fiber optic bronchoscope and said supraglottic airway from said patient, .
